(19) United States
(12) Patent Application Publication
(10) Pub. No.: US 2021/0070858 A1
(43) Pub. Date: Mar. 11, 2021

(54) CD55-Binding Agent-Related Methods and Compositions

(71) Applicant: AgonOx, Inc., Portland, OR (US)

(72) Inventors: Ryan D. Montler, Camas, WA (US); Andrew D. Weinberg, Portland, OR (US); Nicholas P. Morris, Portland, OR (US)

(21) Appl. No.: 16/640,331

(22) PCT Filed: Aug. 21, 2018
(86) PCT No.: PCT/US2018/047356

§ 371 Date: Feb. 19, 2020

Related U.S. Application Data
(60) Provisional application No. 62/548,814, filed on Aug. 22, 2017.

(51) Int. Cl. C07K 16/28; A61P 35/00

(52) U.S. Cl. CPC C07K 16/2809; C07K 16/2818; A61K 2039/507; A61P 35/00; C07K 16/2896

(57) ABSTRACT

Provided are methods of treating cell proliferative disorders, including in some instances, cancer. In certain aspects, provided are methods that include administering to a subject having a cell proliferative disorder a therapeutically effective amount of a CD55-binding agent, where at the time of the administering, abnormally proliferating cells of the cell proliferative disorder are not suspected of exhibiting overexpression of CD55. In some embodiments, provided are methods that include administering to a subject having a cell proliferative disorder a therapeutically effective amount of a CD55-binding agent and a therapeutically effective amount of a T cell activator. T cell activators of interest include, e.g., agonists of co-stimulatory receptors, antagonists of inhibitory signals (e.g., immune checkpoint inhibitors), and the like. Also provided are compositions and kits that find use, e.g., in practicing the methods of the present disclosure.

### CROSS-REFERENCE TO RELATED APPLICATIONS

This application claims the benefit of U.S. Provisional Patent Application No. 62/548,814, filed Aug. 22, 2017, which application is incorporated herein by reference in its entirety.

### INTRODUCTION

T cell-mediated immunity includes multiple sequential steps involving the clonal selection of antigen-specific cells, their activation and proliferation in secondary lymphoid tissues, their trafficking to sites of antigen and inflammation, the execution of direct effector functions and the provision of help (through cytokines and membrane ligands) for a multitude of effector immune cells. Each of these steps is regulated by counterbalancing stimulatory and inhibitory signals that fine-tune the response. Although virtually all inhibitory signals in the immune response ultimately affect intracellular signaling pathways, many are initiated through membrane receptors, the ligands of which are either membrane-bound or soluble (cytokines).

Genetic and epigenetic alterations that are characteristic of all cancers provide a diverse set of antigens that the immune system can use to distinguish tumor cells from their normal counterparts. In the case of T cells, the ultimate amplitude and quality of the response, which is initiated through antigen recognition by the T cell receptor (TCR), is regulated by a balance between co-stimulatory and inhibitory signals (that is, immune checkpoints). Under normal physiological conditions, immune checkpoints are crucial for the maintenance of self-tolerance (that is, the prevention of autoimmunity) and also to protect tissues from damage when the immune system is responding to pathogenic infection.

The expression of immune-checkpoint proteins can be dysregulated by tumors as an important immune resistance mechanism. T cells have been the major focus of efforts to therapeutically manipulate endogenous anti-tumor immunity owing to: their capacity for the selective recognition of peptides derived from proteins in all cellular compartments; their capacity to directly recognize and kill antigen-expressing cells (by CD8+ effector T cells—also known as cytotoxic T lymphocytes (CTLs)); and their ability to orchestrate diverse immune responses (by CD4+ helper T cells), which integrates adaptive and innate effector mechanisms. Thus, agonists of co-stimulatory receptors or antagonists of inhibitory signals, both of which result in the amplification of antigen-specific T cell responses, are agents of particular clinical interest.

CD55 (UniProtKB—P08174 (human); also known as Decay-Accelerating Factor, or “DAF”) is a 70 kDa membrane protein that attaches to the cell membrane via a glycophosphatidylinositol (GPI) anchor. This protein contains four complement control protein (CCP) repeats with a single N-linked glycan positioned between CCP1 and CCP2. CCP2, CCP3, CCP4 and three consecutive lysine residues in a positively charged pocket between CCP2 and CCP3 are involved in its inhibition of the alternate complement pathway. CCP2 and CCP3 alone are involved in its inhibition of the classical pathway.

CD55 recognizes C4b and C3b fragments that condense with cell-surface hydroxyl or amino groups when nascent C4b and C3b are locally generated during C4 and C3 activation. Interaction of CD55 with cell-associated C4b and C3b polypeptides interferes with their ability to catalyze the conversion of C2 and factor B to enzymatically active C2a and Bb, thereby preventing the formation of C4b2a and C3bBb, the amplification convertases of the complement cascade. CD55 further acts as a receptor for coxsackievirus A21, coxsackieviruses B1, B3 and B5, and human echoviruses 6, 7, 11, 12, 20 and 21.

CD55 overexpression has been observed on a variety of human tumor tissues including lung adenocarcinomas and lung squamous cell carcinomas, e.g., see Niehans et al., Am. J. Path. 149:129-142 (1996). U.S. Pat. No. 7,288,249 describes the administration of anti-CD55 antibodies to a subpopulation of cancer patients identified as overexpressing CD55 or expressing a cancer-related variant of CD55. According to the '249 patent, the antibodies administered to the subpopulation of cancer patients find use for antibody-dependent cellular cytotoxicity (ADCC) and may be conjugated to a cytotoxic agent for enhanced killing of cancer cells overexpressing CD55.

### SUMMARY

Provided are methods of treating cell proliferative disorders, including in some instances, cancer. In certain aspects, provided are methods that include administering to a subject having a cell proliferative disorder a therapeutically effective amount of a CD55-binding agent, where at the time of the administering, abnormally proliferating cells of the cell proliferative disorder are not suspected of exhibiting overexpression of CD55. In some embodiments, provided are methods that include administering to a subject having a cell proliferative disorder a therapeutically effective amount of a CD55-binding agent and a therapeutically effective amount of a T cell activator. T cell activators of interest include, e.g., agonists of co-stimulatory receptors, antagonists of inhibitory signals (e.g., immune checkpoint inhibitors), and the like. Also provided are compositions and kits that find use, e.g., in practicing the methods of the present disclosure.

### BRIEF DESCRIPTION OF THE FIGURES

FIG. 1 shows a representative flow cytometric analysis of CD55 expression on CD4 T cells, Treg cells and CD8 T cells in the peripheral blood (top) and primary tumor (bottom) of an HNSCC patient.

FIG. 2, panels A-C, are graphs showing co-stimulation of human T cells via CD55 and CD3.

FIG. 3, panels A-C, are graphs showing the effects of cytokines on CD55/CD3 co-stimulation.

FIG. 4, panels A-E, are graphs showing the levels of cytokine production responsive to CD55/CD3 co-stimulation.

FIG. 5 is a graph showing antigen-specific T cell expansion responsive to CD55/CD3 co-stimulation.

FIG. 6, panels A-C, are graphs illustrating relative CD55 expression and the absence of cross-reactivity across different species.

FIG. 7 shows CD55 protein expression on mouse splenocytes and MCA205 tumor cells from tumor-bearing mice.

FIG. 8 shows CD55 protein expression on mouse hematopoietic cells and tumor cells from MCA205 tumors.

FIG. 9, panels A and B, show survival plots for tumor-bearing mice following administration of anti-CD55 and anti-PD-1 agents.

FIG. 10, panels A-F, are graphs showing the effects of CD55 targeting on human T cell proliferation.

FIG. 11 Costimulation of human CD4 T-cell proliferation. Plate-bound and soluble anti-CD55 antibody assay conditions are shown in panels A and B, respectively.

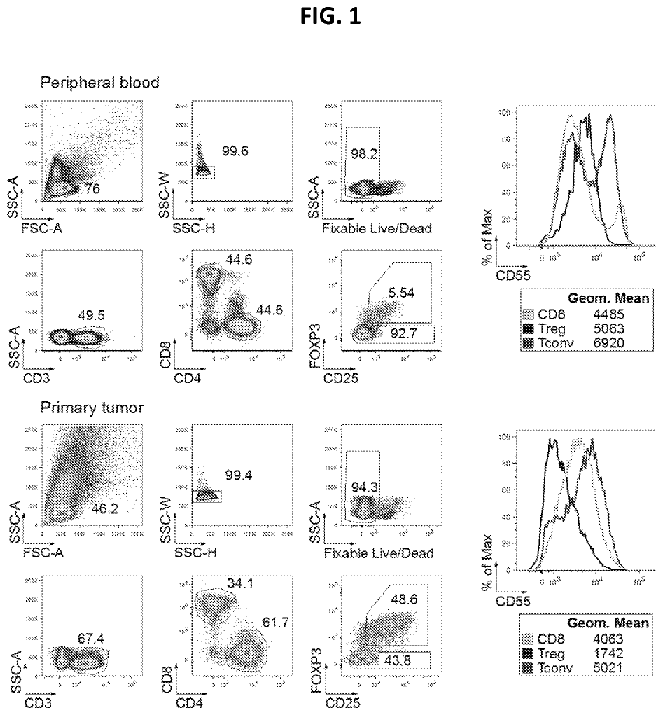

### DETAILED DESCRIPTION

As summarized above, provided are methods of treating cell proliferative disorders, including in some instances, cancer. In certain aspects, provided are methods that include administering to a subject having a cell proliferative disorder a therapeutically effective amount of a CD55-binding agent, where at the time of the administering, abnormally proliferating cells of the cell proliferative disorder are not suspected of exhibiting overexpression of CD55. In some embodiments, provided are methods that include administering to a subject having a cell proliferative disorder a therapeutically effective amount of a CD55-binding agent and a therapeutically effective amount of a T cell activator. T cell activators of interest include, e.g., agonists of co-stimulatory receptors, antagonists of inhibitory signals (e.g., immune checkpoint inhibitors), and the like. Also provided are compositions and kits that find use, e.g., in practicing the methods of the present disclosure.

Before the methods, compositions and kits of the present disclosure are described in greater detail, it is to be understood that the methods, compositions and kits are not limited to particular embodiments described, as such may, of course, vary. It is also to be understood that the terminology used herein is for the purpose of describing particular embodiments only, and is not intended to be limiting, since the scope of the methods, compositions and kits will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods, compositions and kits. The upper and lower limits of these smaller ranges may independently be included in the smaller ranges and are also encompassed within the methods, compositions and kits, subject to any specifically excluded limit in the stated range. Where the stated range includes one or both of the limits, ranges excluding either or both of those included limits are also included in the methods, compositions and kits.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods, compositions and kits belong. Although any methods, compositions and kits similar or equivalent to those described herein can also be used in the practice or testing of the methods, compositions and kits, representative illustrative methods, compositions and kits are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods, compositions and kits are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, compositions and kits, which are, for clarity, described in the context of separate embodiments, may also be provided in combination in a single embodiment. Conversely, various features of the methods, compositions and kits, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods, compositions and kits and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods, compositions and kits. Any recited method can be carried out in the order of events recited or in any other order that is logically possible.

### Methods

As summarized above, the present disclosure provides methods of treating cell proliferative disorders. In some embodiments, the methods include administering to a subject having a cell proliferative disorder a therapeutically effective amount of a CD55-binding agent, where at the time of the administering, abnormally proliferating cells of the cell proliferative disorder are not suspected of exhibiting overexpression of CD55. By “not suspected of exhibiting overexpression of CD55” is meant that CD55 is not suspected to be expressed at higher levels on the abnormally proliferating cells (e.g., cancer cells) compared to a second cell population (e.g., non-abnormally proliferating (e.g., non-cancer) cells of the same tissue type as the abnormally proliferating cells). In some embodiments, at the time of the administering, it has been determined that abnormally proliferating cells of the cell proliferative disorder do not overexpress CD55. In certain aspects, the methods include making such a determination.

In some embodiments, provided are methods that include administering to a subject having a cell proliferative disorder a therapeutically effective amount of a CD55-binding agent, where the CD55-binding agent is administered to the subject to enhance a T cell response to abnormally proliferating cells of the cell proliferative disorder.

The present disclosure relates to the inventors' surprising discovery that CD55-binding agents enhance T cell responses to abnormally proliferating cells of a cell proliferative disorder (e.g., cancer), independent of whether the abnormally proliferating cells overexpress CD55. As such, in some embodiments, the methods of the present disclosure are methods of enhancing a T cell response to abnormally proliferating cells of a cell proliferative disorder, e.g., cancer. Similarly, aspects of the present disclosure include administering a CD55-binding agent to a subject having a cell proliferative disorder, where the purpose of administering the CD55-binding agent is not to induce, but rather to enhance, antibody-dependent cellular cytotoxicity (ADCC). In some embodiments, the methods include administering the CD55-binding agent to an individual receiving an antibody therapy, e.g., an individual receiving an antibody therapy meant to treat a cell proliferative disorder (e.g., cancer) by inducing ADCC, where administering the CD55-binding agent enhances the ADCC of the antibody therapy.

The CD55 binding agent may be administered to any of a variety of subjects. In certain aspects, the subject is a “mammal” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the subject is a human. In certain aspects, the subject is an animal model (e.g., a mouse model, a primate model, or the like) of a cellular proliferative disorder, e.g., cancer.

As summarized above, the subject has a cell proliferative disorder. By “cell proliferative disorder” is meant a disorder wherein unwanted cell proliferation of one or more subset(s) of cells in a multicellular organism occurs, resulting in harm, for example, pain or decreased life expectancy to the organism. Cell proliferative disorders include, but are not limited to, cancer, pre-cancer, benign tumors, blood vessel proliferative disorders (e.g., arthritis, restenosis, and the like), fibrotic disorders (e.g., hepatic cirrhosis, atherosclerosis, and the like), psoriasis, epidermic and dermoid cysts, lipomas, adenomas, capillary and cutaneous hemangiomas, lymphangiomas, nevi lesions, teratomas, nephromas, myofibromatosis, osteoplastic tumors, dysplastic masses, mesangial cell proliferative disorders, and the like.

In some embodiments, the subject has cancer. The subject methods may be employed for the treatment of a large variety of cancers by virtue of the enhanced anti-cancer T cell response achieved. In some embodiments, the subject has a cancer suspected of evading the immune system (e.g., effector T cells), e.g., by co-opting one or more immune checkpoint pathways. “Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. Examples of cancers that may be treated using the subject methods include, but are not limited to, carcinoma, lymphoma, blastoma, and sarcoma. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, prostate cancer, vulval cancer, thyroid cancer, hepatic carcinoma, various types of head and neck cancer, and the like. In certain aspects, the subject has a cancer selected from melanoma, Hodgkin lymphoma, renal cell carcinoma (RCC), bladder cancer, non-small cell lung cancer (NSCLC), and head and neck squamous cell carcinoma (HNSCC). In some embodiments, the subject has a cancer for which administration of a T cell activator (e.g., an agonist of a co-stimulatory receptor, an immune checkpoint inhibitor (e.g., a CTLA-4 inhibitor, a PD-1 inhibitor, or the like)) to treat the cancer (alone or in combination with second anti-cancer agent) has been approved by the Food and Drug Administration (FDA).

As summarized above, in certain aspects, the subject to whom the CD55-binding agent is administered is receiving an antibody therapy. As used herein, “antibody therapy” means that an antibody (which is not the CD55-binding agent) will be, has been, and/or is being administered to the individual for a therapeutic purpose. The antibody therapy will vary depending upon the condition of the individual being treated. In some embodiments, the antibody therapy includes the administration of an antibody (e.g., an IgG, such as an IgG1) that specifically binds to an antigen (e.g., a cell surface antigen, such as a protein or non-protein cell surface antigen) on the surface of a cell relevant to the medical condition of the individual. For example, the antibody administered as part of the antibody therapy may bind to an antigen present on the surface of a cell that contributes to the medical condition (e.g., a cancer cell in a subject having cancer), where binding of the antibody to the antigen reduces or abolishes the cell's contribution to the medical condition. In some embodiments, the subject has cancer, and the antibody administered as part of the antibody therapy is selected from ramucirumab, bevacizumab, nivolumab, pembrolizumab, sipuleucel-T, trastuzumab, pertuzumab, catumaxomab, cetuximab, panitumumab, ipilimumab, alemtuzumab, gemtuzumab, ozogamicin, brentuximab vedotin, rituximab, ofatumumab, ⁹⁰Y-ibritumomab, ¹³¹I-tositumomab, blinatumomab, imatinib, dasatinib, nilotinib, bosutinib, ponatinib, antigen-binding variants (including fragments) thereof, and any combination thereof.

Any of the CD55-binding agents described herein may specifically bind to CD55. By “specifically binds” is meant the CD55-binding agent exhibits a binding affinity to CD55 such that the CD55-binding agent has an equilibrium binding constant (K_{D}) for CD55 of less than or equal to about 10⁻⁵ M, less than or equal to about 10⁻⁶ M, less than or equal to about 10⁻⁷ M, less than or equal to about 10⁻⁸ M, less than or equal to about 10⁻⁹ M, 10⁻¹⁰ M, 10⁻¹¹ M, or 10⁻¹² M or less. Such affinities may be readily determined using conventional techniques, such as by equilibrium dialysis, surface plasmon resonance (SPR) technology (e.g., using the BIAcore 2000 instrument, using general procedures outlined by the manufacturer), radioimmunoassay, or by another method.

Methods are available for measuring the affinity of a candidate CD55-binding agent for CD55 expressed on the surface of cells (e.g., T cells) using direct binding or competition binding assays. In a direct binding assay, the equilibrium binding constant (K_{D}) may be measured using a candidate CD55-binding agent conjugated to a fluorophore or radioisotope, or a candidate CD55-binding agent that contains an N- or C-terminal epitope tag for detection by a labeled antibody. If labels or tags are not feasible or desired, a competition binding assay can be used to determine the half-maximal inhibitory concentration (IC₅₀), the amount of unlabeled candidate CD55-binding agent at which 50% of the maximal signal of the labeled competitor is detectable. A K_{D} value can then be calculated from the measured IC₅₀ value. Ligand depletion will be more pronounced when measuring high-affinity interactions over a lower concentration range, and can be avoided or minimized by decreasing the number of cells added in the experiment or by increasing the binding reaction volumes.

The CD55-binding agent administered to the subject may vary. In certain aspects, the CD55-binding agent is a small molecule. As used herein, a “small molecule” is a compound having a molecular weight of 1000 atomic mass units (amu) or less. In some embodiments, the small molecule is 750 amu or less, 500 amu or less, 400 amu or less, 300 amu or less, or 200 amu or less. In certain aspects, the small molecule is not made of repeating molecular units such as are present in a polymer. In some embodiments, the CD55-binding agent is a small molecule known to bind CD55. In other aspects, a small molecule CD55-binding agent is identified, e.g., using a suitable approach for screening small molecules, e.g., by screening a combinatorial library of small molecules.

In some embodiments, the CD55-binding agent is a peptide or polypeptide. When the CD55-binding agent is a peptide or polypeptide, the agent may be a CD55 ligand. CD55 ligands of interest include, but are not limited to, complement component 3B (C3B), complement component 4B (C4B), or CD55-binding derivatives thereof (e.g., non-functional C3B or C4B derivatives thereof, such as derivatives that are non-functional in the classical complement pathway, alternate complement pathway, and/or lectin pathway), including CD55-binding fragments thereof. In some embodiments, the CD55 ligand is a viral protein that binds to CD55 (e.g., a CD55-binding protein of coxsackievirus A21, coxsackieviruses B1, B3 and B5, human echoviruses 6, 7, 11, 12, 20 and 21, or the like), or CD55-binding derivatives thereof, including CD55-binding fragments thereof. CD55-binding viral protein derivatives may include modifications that, e.g., reduce or eliminate immunogenicity that a wild-type protein may exhibit in the subject.

In certain aspects, the CD55-binding agent is an antibody that specifically binds CD55. The terms “antibody” and “immunoglobulin” include antibodies or immunoglobulins of any isotype (e.g., IgG (e.g., IgG1, IgG2, IgG3 or IgG4), IgE, IgD, IgA, IgM, etc.), whole antibodies (e.g., antibodies composed of a tetramer which in turn is composed of two dimers of a heavy and light chain polypeptide); single chain antibodies; fragments of antibodies (e.g., fragments of whole or single chain antibodies) which retain specific binding to CD55, including, but not limited to, Fv, single chain Fv (scFv), Fab, F(ab′)₂, Fab′, (scFv′)₂, and diabodies; chimeric antibodies; monoclonal antibodies, human antibodies, humanized antibodies (e.g., humanized whole antibodies, humanized antibody fragments, etc.); and fusion proteins including an antigen-binding portion of an antibody and a non-antibody protein or fragment thereof, e.g., an antibody Fc region or fragment thereof. The antibodies may be detectably labeled, e.g., with an in vivo imaging agent, or the like. The antibodies may be further conjugated to other moieties, such as, e.g., polyethylene glycol (PEG), etc. Fusion to an antibody Fc region (or a fragment thereof), conjugation to PEG, etc. may find use, e.g., for increasing serum half-life of the antibody upon administration to the subject.

The CD55-binding agent may be a known CD55-binding agent. For example, in some embodiments, the antibody is the JS-11 antibody (BioLegend®) or other antibody known to specifically bind CD55. In some embodiments, the CD55-binding agent is an antibody that competes for binding to CD55 with the JS-11 antibody or other antibody known to specifically bind human CD55. Such antibodies may be identified using any of the various suitable competitive binding assays known in the art.

In any of the methods, compositions and kits of the present disclosure, the CD55-binding agent (e.g., small molecule, anti-CD55 antibody, ligand, etc.) may be a CD55 agonist. An example assay for determining whether a CD55 binding agent is a CD55 agonist is described in Example 7. In that assay, costimulation of CD4 T cells is assessed for a plate-bound CD55-binding agent and the corresponding soluble CD55-binding agent. Costimulation by the agent when plate-bound but not when soluble (or greater costimulation by the agent when plate-bound as compared to when soluble) indicates that the agent is an agonist, e.g., because the plate-bound agent is able to concentrate CD55 molecules on the surface of the cell, thereby facilitating signaling through CD55.

In certain aspects, the CD55-binding agent is identified, e.g., using a suitable approach for screening small molecules (e.g., by screening a combinatorial library of small molecules), antibodies (e.g., by phage or yeast display of antibody libraries), ligands, or the like for the ability to bind CD55.

Antibodies that specifically bind CD55 can be prepared using a wide variety of techniques known in the art including the use of hybridoma, recombinant, phage display technologies, or a combination thereof. For example, an antibody may be made and isolated using methods of phage display. Phage display is used for the high-throughput screening of protein interactions. Phages may be utilized to display antigen-binding domains expressed from a repertoire or combinatorial antibody library (e.g., human or murine). Phage expressing an antigen binding domain that binds CD55 can be selected or identified with CD55, e.g., using labeled CD55 bound or captured to a solid surface or bead. Phage used in these methods are typically filamentous phage including fd and M13 binding domains expressed from phage with Fab, Fv (individual Fv region from light or heavy chains) or disulfide stabilized Fv antibody domains recombinantly fused to either the phage gene III or gene VIII protein. Exemplary methods are set forth, for example, in U.S. Pat. No. 5,969,108, Hoogenboom, H. R. and Chames, Immunol. Today 2000, 21:371; Nagy et al. Nat. Med. 2002, 8:801; Huie et al., Proc. Natl. Acad. Sci. USA 2001, 98:2682; Lui et al., J. Mol. Biol. 2002, 315:1063, each of which is incorporated herein by reference. Several publications (e.g., Marks et al., Bio/Technology 1992, 10:779-783) have described the production of high affinity human antibodies by chain shuffling, as well as combinatorial infection and in vivo recombination as a strategy for constructing large phage libraries. In another embodiment, ribosomal display can be used to replace bacteriophage as the display platform (see, e.g., Hanes et al., Nat. Biotechnol. 2000, 18:1287; Wilson et al., Proc. Natl. Acad. Sci. USA 2001, 98:3750; or Irving et al., J. Immunol. Methods 2001, 248:31). Cell surface libraries may be screened for antibodies (Boder et al., Proc. Natl. Acad. Sci. USA 2000, 97:10701; Daugherty et al., J. Immunol. Methods 2000, 243:211). Such procedures provide alternatives to traditional hybridoma techniques for the isolation and subsequent cloning of monoclonal antibodies.

After phage selection, the antibody coding regions from the phage can be isolated and used to generate whole antibodies, including human antibodies, or any desired antigen binding fragment, and expressed in any desired host, including mammalian cells, insect cells, plant cells, yeast, and bacteria. For example, techniques to recombinantly produce Fv, scFv, Fab, F(ab′)₂, and Fab′ fragments may be employed using methods known in the art.

In some embodiments, provided are methods that include administering a combination of the CD55-binding agent and a T cell activator. According to such methods, the administering is to a subject having a cell proliferative disorder. Optionally, at the time of the administering (e.g., the initial administration of the CD55-binding agent, the T cell activator, or both (if present in a single formulation)), abnormally proliferating cells of the cell proliferative disorder are not suspected of exhibiting overexpression of CD55.

As used herein, a “T cell activator” is an agent that stimulates an immune response in a T cell or group of T cells. Such an immune response involves the engagement of the T cell receptor (TCR), present on the surface of a T cell, with a small peptide antigen non-covalently presented on the surface of an antigen presenting cell (APC) by a major histocompatibility complex (MHC; also referred to in humans as a human leukocyte antigen (HLA) complex). This engagement represents the immune system's targeting mechanism and is a requisite molecular interaction for T cell activation and effector function. Following epitope-specific cell targeting, the targeted T cells are activated through engagement of costimulatory proteins found on the APC with counterpart costimulatory proteins on the T cells. Both signals—epitope/TCR binding and engagement of APC costimulatory proteins with T cell costimulatory proteins—are required to drive T cell specificity and activation. The TCR is specific for a given epitope; however, the costimulatory protein is not epitope specific and instead is generally expressed on all T cells or on large T cell subsets.

Various assays can be utilized in order to determine whether an immune response has been stimulated in a T cell or group of T cells, i.e., whether a T cell or group of T cells has become “activated”. In certain aspects, stimulation of an immune response in T cells can be determined by measuring antigen-induced production of cytokines by T cells. In some embodiments, stimulation of an immune response in T cells can be determined by measuring antigen-induced production of IFNγ, IL-4, IL-2, IL-10, IL-17 and/or TNFα by T cells. In some embodiments, antigen-produced production of cytokines by T cells can be measured by intracellular cytokine staining followed by flow cytometry. In some embodiments, antigen-induced production of cytokines by T cells can be measured by surface capture staining followed by flow cytometry. In some embodiments, antigen-induced production of cytokines by T cells can be measured by determining cytokine concentration in supernatants of activated T cell cultures. In some embodiments, this can be measured by ELISA.

In some embodiments, antigen-produced production of cytokines by T cells can be measured by ELISPOT assay. In general, ELISPOT assays employ a technique very similar to the sandwich enzyme-linked immunosorbent assay (ELISA) technique. An antibody (e.g. monoclonal antibody, polyclonal antibody, etc.) is coated aseptically onto a PVDF (polyvinylidene fluoride)-backed microplate. Antibodies are chosen for their specificity for the cytokine in question. The plate is blocked (e.g. with a serum protein that is non-reactive with any of the antibodies in the assay). Cells of interest are plated out at varying densities, along with antigen or mitogen, and then placed in a humidified 37° C. CO2 incubator for a specified period of time. Cytokine secreted by activated cells is captured locally by the coated antibody on the high surface area PVDF membrane. After washing the wells to remove cells, debris, and media components, a secondary antibody (e.g., a biotinylated polyclonal antibody) specific for the cytokine is added to the wells. This antibody is reactive with a distinct epitope of the target cytokine and thus is employed to detect the captured cytokine. Following a wash to remove any unbound biotinylated antibody, the detected cytokine is then visualized using an avidin-HRP, and a precipitating substrate (e.g., AEC, BCIP/NBT). The colored end product (a spot, usually a blackish blue) typically represents an individual cytokine-producing cell. Spots can be counted manually (e.g., with a dissecting microscope) or using an automated reader to capture the microwell images and to analyze spot number and size. In some embodiments, each spot correlates to a single cytokine-producing cell.

In some instances, T cells activated by the T cell activator are specific for an epitope present on abnormally proliferative cells underlying the cellular proliferative disorder (e.g., cancer cells in a subject having cancer), and contacting such T cells with the T cell activator increases cytotoxic activity of the T cells toward the abnormally proliferating cells, increases the number of such epitope-specific T cells, or a combination thereof.

A wide variety of known agents may be employed as the T cell activator. In some embodiments, the T cell activator is an immune checkpoint inhibitor. As used herein, an “immune checkpoint inhibitor” is any agent (e.g., small molecule, nucleic acid, protein (e.g., antibody)) that prevents the suppression of any component in the immune system such as MHC class presentation, T cell presentation and/or differentiation, any cytokine, chemokine or signaling for immune cell proliferation and/or differentiation. In certain aspects, the immune checkpoint inhibitor is an agonist of a T cell co-stimulatory receptor. Non-limiting examples of such co-stimulatory receptors include CD28, ICOS, CD137, OX40, CD27, and the like. In some embodiments, the immune checkpoint inhibitor is an antagonist of a T cell inhibitory signal. The T cell inhibitory signal may be a signal transmitted through, e.g., PD-1, PD-L1, CTLA4, BTLA, KIR, LAG-3, TIM-3, A2aR, or the like, and any combinations thereof.

In some embodiments, the T cell activator is an immune checkpoint inhibitor selected from a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, an OX40 agonist, a glucocorticoid-induced TNFR-related protein (GITR) agonist, a CD137 agonist, a CD40 agonist, and any combination thereof.

In certain aspects, the T cell activator is a cytokine. Cytokines of interest in the context of the present disclosure are those that promote T cell activation (e.g., IL-1, and the like), promote proliferation of activated T cells (e.g., IL-2, and the like), etc. Non-limiting examples of cytokines that may be administered with the CD55-binding agent include IL-2, IL-4, IL-15, and any combination thereof.

In some embodiments, the T cell activator is an antagonist of an inhibitory immune receptor. In certain aspects, such an antagonist binds directly to the inhibitory immune receptor, thereby blocking activation of the inhibitory immune receptor, e.g., by preventing binding of the receptor to its ligand. In other aspects, such an antagonist binds to the ligand of an inhibitory immune receptor, thereby blocking activation of the inhibitory immune receptor by preventing binding of the receptor to its ligand. Antagonists of inhibitory immune receptors that may be administered with the CD55-binding agent include, but are not limited to, TGF-β.

The CD55-binding agent and, if also administered, the T cell activator, are administered in a therapeutically effective amount. By “therapeutically effective amount” is meant a dosage sufficient to produce a desired result, e.g., an amount sufficient to effect beneficial or desired therapeutic (including preventative) results, such as a reduction in a symptom of the proliferative disorder, as compared to a control. When the cell proliferative disorder is cancer, in some embodiments, the therapeutically effective amount is sufficient to slow the growth of a tumor, reduce the size of a tumor, and/or the like. An effective amount can be administered in one or more administrations.

When the methods include administering a combination of the CD55-binding agent and a T cell activator, the CD55-binding agent and the T cell activator may be administered concurrently (e.g., in the same or separate formulations), sequentially, or both. For example, according to certain embodiments, the T cell activator is administered to the subject prior to administration of the CD55-binding agent, concurrently with administration of the CD55-binding agent, or both. In some embodiments, the CD55-binding agent is administered to the subject prior to administration of the T cell activator, concurrently with administration of the T cell activator, or both.

In certain aspects, the one or more agents are administered according to a dosing regimen approved for individual use. In some embodiments, the administration of the CD55-binding agent permits the T cell activator to be administered according to a dosing regimen that involves one or more lower and/or less frequent doses, and/or a reduced number of cycles as compared with that utilized when the T cell activator is administered without administration of the CD55-binding agent. In some embodiments, the administration of the T cell activator permits the CD55-binding agent to be administered according to a dosing regimen that involves one or more lower and/or less frequent doses, and/or a reduced number of cycles as compared with that utilized when the CD55-binding agent is administered without administration of the T cell activator.

As noted above, in certain aspects, one or more doses of the CD55-binding agent and T cell activator are administered at the same time; in some such embodiments, such agents may be administered present in the same pharmaceutical composition. In some embodiments, however, the CD55-binding agent and T cell activator are administered to the subject in different compositions and/or at different times. For example, the CD55-binding agent may be administered prior to administration of the T cell activator (e.g., in a particular cycle). Alternatively, the T cell activator may be administered prior to administration of the CD55-binding agent (e.g., in a particular cycle). The second agent to be administered may be administered a period of time that starts at least 1 hour, 3 hours, 6 hours, 12 hours, 24 hours, 48 hours, 72 hours, or up to 5 days or more after the administration of the first agent.

In one example, the CD55-binding agent is administered to the subject for a desirable period of time prior to administration of the T cell activator. In certain aspects, such a regimen “primes” the immune system for T cell activation by the T cell activator. In another example, the T cell activator is administered to the subject for a desirable period of time prior to administration of the CD55-binding agent. In certain aspects, such a regimen “primes” the immune system for T cell activation by the CD55-binding agent.

In some embodiments, administration of one agent is specifically timed relative to administration of another agent. For example, in some embodiments, a first agent is administered so that a particular effect is observed (or expected to be observed, for example based on population studies showing a correlation between a given dosing regimen and the particular effect of interest).

In certain aspects, desired relative dosing regimens for agents administered in combination may be assessed or determined empirically, for example using ex vivo, in vivo and/or in vitro models; in some embodiments, such assessment or empirical determination is made in vivo, in a patient population (e.g., so that a correlation is established), or alternatively in a particular subject of interest.

By way of example, the CD55-binding agent may be administered a period of time after administration of the T cell activator. The period of time may be selected to be correlated with an increase in T cell activation by the T cell activator. In certain aspects, the relevant period of time permits (e.g., is correlated with) T cell activation that is 10% or more, 20% or more, 30% or more, 40% or more, 50% or more, 60% or more, 70% or more, 80% or more, 90% or more, 100% or more, 150% or more, 200% or more, 250% or more, 300% or more, 350% or more, 400% or more, 450% or more, or 500% or more, than that observed prior to (or at the moment of) the administration of the T cell activator.

In some embodiments, the CD55-binding agent and T cell activator are administered according to an intermittent dosing regimen including at least two cycles. Where two or more agents are administered in combination, and each by such an intermittent, cycling, regimen, individual doses of different agents may be interdigitated with one another. In certain aspects, one or more doses of the second agent is administered a period of time after a dose of the first agent. In some embodiments, each dose of the second agent is administered a period of time after a dose of the first agent. In certain aspects, each dose of the first agent is followed after a period of time by a dose of the second agent. In some embodiments, two or more doses of the first agent are administered between at least one pair of doses of the second agent; in certain aspects, two or more doses of the second agent are administered between al least one pair of doses of the first agent. In some embodiments, different doses of the same agent are separated by a common interval of time; in some embodiments, the interval of time between different doses of the same agent varies. In certain aspects, different doses of the different agents are separated from one another by a common interval of time; in some embodiments, different doses of the different agents are separated from one another by different intervals of time.

One exemplary protocol for interdigitating two intermittent, cycled dosing regimens (e.g., for potentiating the effect of the CD55-binding agent, the T cell activator, or both), may include: (a) a first dosing period during which a therapeutically effective amount a first agent is administered to a subject; (b) a first resting period; (c) a second dosing period during which a therapeutically effective amount of a second agent and, optionally, a third agent, is administered to the subject; and (d) a second resting period.

In some embodiments, the first resting period and second resting period may correspond to an identical number of hours or days. Alternatively, in some embodiments, the first resting period and second resting period are different, with either the first resting period being longer than the second one or, vice versa. In some embodiments, each of the resting periods corresponds to 120 hours, 96 hours, 72 hours, 48 hours, 24 hours, 12 hours, 6 hours, 30 hours, 1 hour, or less. In some embodiments, if the second resting period is longer than the first resting period, it can be defined as a number of days or weeks rather than hours (for instance 1 day, 3 days, 5 days, 1 week, 2, weeks, 4 weeks or more).

If the first resting period's length is determined by existence or development of a particular biological or therapeutic event (e.g., increased T cell activation), then the second resting period's length may be determined on the basis of different factors, separately or in combination. Exemplary such factors may include type and/or stage of a cancer against which the agents are administered; identity and/or properties (e.g., pharmacokinetic properties) of the first agent, and/or one or more features of the patient's response to therapy with the first agent. In some embodiments, length of one or both resting periods may be adjusted in light of pharmacokinetic properties (e.g., as assessed via plasma concentration levels) of one or the other (or both) of the administered agents. For example, a relevant resting period might be deemed to be completed when plasma concentration of the relevant agent is below about 1 μg/ml, 0.1 μg/ml, 0.01 μg/ml or 0.001 μg/ml, optionally upon evaluation or other consideration of one or more features of the subject's response.

In certain aspects, the number of cycles for which a particular agent is administered may be determined empirically. Also, in some embodiments, the precise regimen followed (e.g., number of doses, spacing of doses (e.g., relative to each other or to another event such as administration of another therapy), amount of doses, etc.) may be different for one or more cycles as compared with one or more other cycles.

The CD55-binding agent, and if also administered, a T cell activator, may be administered via a route of administration independently selected from oral, parenteral (e.g., by intravenous, intra-arterial, subcutaneous, intramuscular, or epidural injection), topical, or nasal administration. According to certain embodiments, the CD55-binding agent and a T cell activator are both administered parenterally, either concurrently (in the same pharmaceutical composition or separate pharmaceutical compositions) or sequentially.

As described above, the subject methods are for treating a cell proliferative disorder (e.g., cancer). By treatment is meant at least an amelioration of one or more symptoms associated with the cell proliferative disorder of the subject, where amelioration is used in a broad sense to refer to at least a reduction in the magnitude of a parameter, e.g. symptom, associated with the cell proliferative disorder (e.g., cancer) being treated. As such, treatment also includes situations where the cell proliferative disorder, or at least one or more symptoms associated therewith, are completely inhibited, e.g., prevented from happening, or stopped, e.g., terminated, such that the subject no longer suffers from the cell proliferative disorder, or at least the symptoms that characterize the cell proliferative disorder.

### Compositions

As summarized above, aspects of the present disclosure include pharmaceutical compositions. In some embodiments, a pharmaceutical composition of the present disclosure includes a CD55-binding agent (e.g., any of the CD55-binding agents described above), and a pharmaceutically acceptable excipient. In certain aspects, a pharmaceutical composition of the present disclosure includes a CD55-binding agent (e.g., any of the CD55-binding agents described above), a T cell activator (e.g., any of the T cell activators described above), and a pharmaceutically acceptable excipient.

As will be appreciated, the pharmaceutical compositions of the present disclosure may include any of the agents and features described above in the section relating to the subject methods, which are not reiterated in detail herein for purposes of brevity.

In some embodiments, the CD55-binding agent present in the pharmaceutical composition is a small molecule. In certain aspects, the CD55-binding agent present in the pharmaceutical composition is a peptide or polypeptide. When the CD55-binding agent is a peptide or polypeptide, the CD55-binding agent may be a CD55 ligand, an antibody that specifically binds CD55, or the like. When the CD55-binding agent is an antibody, the antibody may be selected from an IgG, Fv, scFv, Fab, F(ab′)2, and Fab′.

When a pharmaceutical composition of the present disclosure includes a T cell activator, in certain aspects, the T cell activator is an immune checkpoint inhibitor. Immune checkpoint inhibitors of interest include, but are not limited to, an agonist of a T cell co-stimulatory receptor, an antagonist of a T cell inhibitory signal, and/or the like. In some embodiments, the immune checkpoint inhibitor is selected from a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, an OX40 agonist, a glucocorticoid-induced TNFR-related protein (GITR) agonist, a CD137 agonist, and a CD40 agonist.

When a pharmaceutical composition of the present disclosure includes a T cell activator, in certain aspects, the T cell activator is a cytokine. In some embodiments, when a pharmaceutical composition of the present disclosure includes a T cell activator, the T cell activator is an antagonist of an inhibitory immune receptor. In certain aspects, a pharmaceutical composition of the present disclosure includes a combination (that is, two or more) of any of the T cell activators described herein.

The CD55-binding agent, T cell activator, or both, can be incorporated into a variety of formulations for therapeutic administration. More particularly, the agent(s) (that is, CD55-binding agent and/or T cell activator) can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the agents for administration to the subject (e.g., suitable for human administration) are generally sterile and may further be free of detectable pyrogens or other contaminants contraindicated for administration to a patient according to a selected route of administration.

In pharmaceutical dosage forms, the agent(s) can be administered in the form of their pharmaceutically acceptable salts, or they may also be used alone or in appropriate association, as well as in combination, with other pharmaceutically active compounds. The following methods and carriers/excipients are merely examples and are in no way limiting.

For oral preparations, the agent(s) can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agent(s) can be formulated for parenteral (e.g., intravenous, intra-arterial, intraosseous, intramuscular, intracerebral, intracerebroventricular, intrathecal, subcutaneous, etc.) administration. In certain aspects, the agent(s) are formulated for injection by dissolving, suspending or emulsifying the agent(s) in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include the agent(s) may be prepared by mixing the agent(s) having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the agent(s) may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/v.

A lyoprotectant may also be added in order to protect the CD55-binding agent and/or T cell activator against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included in an amount of about 10 mM to 500 nM.

In some embodiments, the pharmaceutical composition includes the CD55-binding agent and/or T cell activator, and one or more of the above-identified components (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% (w/v).

### Kits

As summarized above, the present disclosure provides kits. The kits find use, e.g., in practicing the methods of the present disclosure. In some embodiments, a subject kit includes a pharmaceutical composition that includes a CD55-binding agent (e.g., any of the CD55-binding agents described above). In certain aspects, provided are kits that include any of the pharmaceutical compositions described herein, including any of the pharmaceutical compositions described above in the section relating to the compositions of the present disclosure. Kits of the present disclosure may include instructions for administering the pharmaceutical composition to a subject having a cell proliferative disorder, e.g., cancer. In some embodiments, a kit of the present disclosure includes instructions for administering the pharmaceutical composition to a subject having a cell proliferative disorder in which abnormally proliferating cells of the cell proliferative disorder are not suspected of exhibiting overexpression of CD55. In some embodiments, a kit of the present disclosure includes instructions for administering the pharmaceutical composition to a subject having a cell proliferative disorder to enhance a T cell response to abnormally proliferating cells of the cell proliferative disorder. In certain aspects, a kit of the present disclosure includes instructions for administering the pharmaceutical composition to a subject receiving an antibody therapy. In some embodiments, the antibody therapy is being administered to the subject to treat the cell proliferative disorder by inducing antibody-dependent cellular cytotoxicity (ADCC), and the instructions are for administering the CD55-binding to the subject to potentiate ADCC of the antibody therapy.

In some embodiments, kits are provided that include a CD55-binding agent, a T cell activator, and instructions for administering the CD55 binding agent and T cell activator to a subject having a cell proliferative disorder, e.g., cancer. The instructions may further include any of the instructions for the kits described above, e.g., for administering the pharmaceutical composition to a subject having a cell proliferative disorder to enhance a T cell response to abnormally proliferating cells of the cell proliferative disorder (e.g., cancer), for administering the pharmaceutical composition to a subject receiving an antibody therapy, and/or the like. The CD55-binding agent and T cell activator may be present in the same container, or may be present in separate containers.

The subject kits may include a quantity of the compositions, present in unit dosages, e.g., ampoules, or a multi-dosage format. As such, in certain embodiments, the kits may include one or more (e.g., two or more) unit dosages (e.g., ampoules) of a composition that includes a CD55-binding agent, a T cell activator, or both. The term “unit dosage”, as used herein, refers to physically discrete units suitable as unitary dosages for human and animal subjects, each unit containing a predetermined quantity of the composition calculated in an amount sufficient to produce the desired effect. The amount of the unit dosage depends on various factors, such as the particular CD55-binding agent and/or T cell activator employed, the effect to be achieved, and the pharmacodynamics associated with the CD55-binding agent and/or T cell activator, in the subject. In yet other embodiments, the kits may include a single multi dosage amount of the composition.

As will be appreciated, the kits of the present disclosure may include any of the agents and features described above in the section relating to the subject methods and compositions, which are not reiterated in detail herein for purposes of brevity.

In some embodiments, the CD55-binding agent present in the kit is a small molecule. In certain aspects, the CD55-binding agent present in the kit is a peptide or polypeptide. When the CD55-binding agent is a peptide or polypeptide, the CD55-binding agent may be a CD55 ligand, an antibody that specifically binds CD55, or the like. When the CD55-binding agent is an antibody, the antibody may be selected from an IgG, Fv, scFv, Fab, F(ab′)2, and Fab′.

When a kit of the present disclosure includes a T cell activator, in certain aspects, the T cell activator is an immune checkpoint inhibitor. Immune checkpoint inhibitors of interest include, but are not limited to, an agonist of a T cell co-stimulatory receptor, an antagonist of a T cell inhibitory signal, and/or the like. In some embodiments, the immune checkpoint inhibitor is selected from a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, an OX40 agonist, a glucocorticoid-induced TNFR-related protein (GITR) agonist, a CD137 agonist, and a CD40 agonist. When a kit of the present disclosure includes a T cell activator, in certain aspects, the T cell activator is a cytokine. In some embodiments, when a kit of the present disclosure includes a T cell activator, the T cell activator is an antagonist of an inhibitory immune receptor. In certain aspects, a kit of the present disclosure includes a combination (that is, two or more) of any of the T cell activators described herein.

Components of the kits may be present in separate containers, or multiple components may be present in a single container. A suitable container includes a single tube (e.g., vial), ampoule, one or more wells of a plate (e.g., a 96-well plate, a 384-well plate, etc.), or the like.

The instructions (e.g., instructions for use (IFU)) included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, DVD, CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, the means for obtaining the instructions is recorded on a suitable substrate.

The following examples are offered by way of illustration and not by way of limitation.

### EXPERIMENTAL

### Example 1—Expression of CD55 on T Cells in the Blood and Primary Tumor of an HNSCC Patient

Paired peripheral blood and primary tumor cells from an HNSCC patient were thawed and stained for the expression of CD55 on different T cell populations. T cells were identified by the expression of CD3 and further gated on CD4 and CD8 T cells. Treg cells were identified by the coexpression of CD25 and FOXP3 on CD4 T cells. Tconv were CD4 T cells that did not coexpress CD25 and FOXP3. Numbers above outlined area indicate percent gated cells. The expression of CD55 on Tconv, Treg cells and CD8 T cells is shown as an overlay for all three T cell populations. The geometric mean for CD55 expression for each cell population is indicated below the histogram. These results showed that a subset of Tconv and CD8 T cells expressed high levels of CD55 whereas the remaining cells displayed low expression of CD55 in the peripheral blood. Interestingly, Treg cells expressed intermediate levels of CD55. In contrast, the expression levels of CD55 in the tumor were reduced for Tconv when compared to the blood. This effect was even more pronounced for Treg cells. As for CD8 T cells, cells expressing high levels of CD55 in the blood were absent in the tumor while the expression of CD55 on the remaining CD8 T cells was unchanged when compared to the blood. Thus, expression of CD55 on T cells varies depending on the T cell population analyzed and this might have an implication in regards to which cells are targeted by the anti-CD55 antibody in vivo.

### Example 2—Co-Stimulation of Human T Cells Via CD55 and CD3

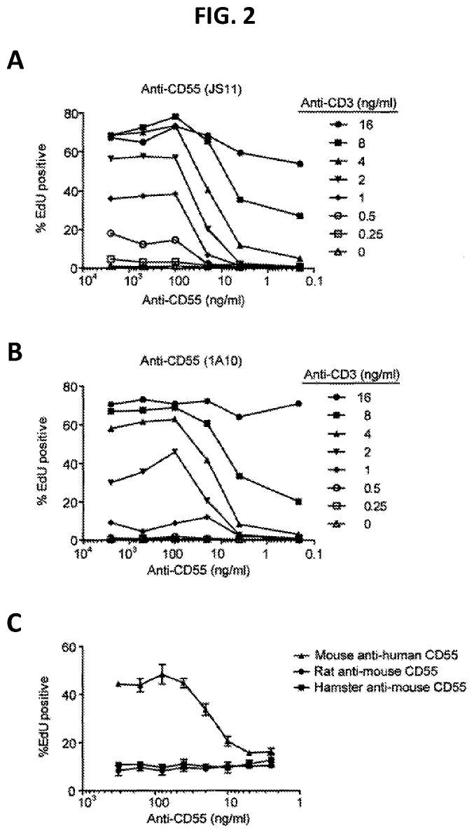

The binding agents to TCR (such as anti-CD3 antibodies) and to a potential co-stimulation target may be provided in tandem to purified T cells. The resulting T cell proliferation may be quantified and used to gauge the efficacy of the co-stimulation target. FIG. 2, panels A-C are graphs depicting the results of T cell co-stimulation assays using anti-CD3 along with different CD55-binding agents. FIG. 2, panels A and B, illustrate the co-stimulatory effects on effector CD4+ T cell proliferation using two different anti-CD55 antibodies, JS11 and 1A10, respectively.

CD4+ T cells were purified from frozen PBMCs and resuspended in RPMI media supplemented with penicillin, streptomycin, glutamine, non-essential amino acids, HEPES buffer, and sodium pyruvate. PHA-L (2 μg/ml) and IL2 (60 IU/ml) were added to the media to activate the cells, which were then incubated at 37° under 5% CO2 for 96 hours. Ninety-six-well plates were coated with goat anti-mouse IgG and then blocked with BSA. Mouse anti-human CD3 (OKT3) was prepared at 16 ng/ml in 1% BSA/DPBS, and added to the plates in serial, two-fold dilutions. Mouse anti-human CD55 was then added in serial, five-fold dilutions between 2500 ng/ml and 0.2 ng/ml to create a serial matrix of anti-CD3 and anti-CD55. Activated CD4+ T cells were then added to each well at 50,000 cells/well in 200 μl of buffer, and then incubated at 37° for 48 hours. Cells were then labeled with 5-ethynyl-2′-deoxyuridine (EdU) at a final concentration of 10 μM) using the Click-it DNA labeling kit (Invitrogen) as per the manufacturer's instructions for 18 hours. Cells were then treated with live-dead stain (E450; eBiosciences) and developed using the Alex647 agent (Invitrogen). Proliferating cells were then quantified via flow cytometry.

As shown in FIG. 2, panel A, the JS11 anti-CD55 clone stimulated proliferation of the CD4+ T cells in a dose-dependent manner, with proliferation observed at concentrations as low as 4 ng/ml of anti-CD55 with relatively high concentrations of anti-CD3, and at concentrations as low as 20 ng/ml of anti-CD55 for more standard anti-CD3 concentrations (1-2 ng/ml). As shown in FIG. 2, panel B, the 1A10 anti-CD55 clone also stimulated proliferation of the CD4+ T cells in a dose-dependent manner, though the efficacy was lower than for the JS11 clone. For 1A10, no proliferation was seen for concentrations of anti-CD3 below 1 ng/ml with any concentration of anti-CD55. These results indicate that both the JS11 and 1A10 CD55-binding agent successfully demonstrated dose-dependent co-stimulation/stimulation (mitogenesis) of the CD4+ T cells when co-cultured with anti-CD3. These results suggest that co-stimulation with a CD55 binding agent increases T cell proliferation.

To evaluate species specificity of anti-CD55 in co-stimulation, experiments were performed as above, except anti-CD3 was added at 2 ng/ml to all wells, and mouse anti-human CD55, rat anti-mouse CD55 and hamster anti-mouse CD55 were added in serial two-fold dilutions between 320 ng/ml and 2.5 ng/ml. As shown in FIG. 2, panel C, no dose-dependent co-stimulation was observed for either anti-mouse CD55 antibody.

### Example 3—Cytokine Effects on T Cell Co-Stimulation with CD55-Binding Agents

To further characterize the in vitro function of CD55 binding agents, cytokine effects on T cell co-stimulation with anti-CD55 were analyzed. More specifically, either IL-12 or TGF-β were added to culture media in CD3/CD55 co-stimulation assays. IL-12 is secreted by dendritic cells, macrophages, and neutrophils and binds IL-12R, found on activated T cells. Binding of IL-12 to IL-12R stimulates the JAK-STAT pathway, and is associated with promotion of TH1 cell development. In contrast, TGF-β is secreted by Treg cells, represses proliferation of both CD4+ and CD8+ T cells, and may be a primary means by which a tumor evades immunogenic regulation.

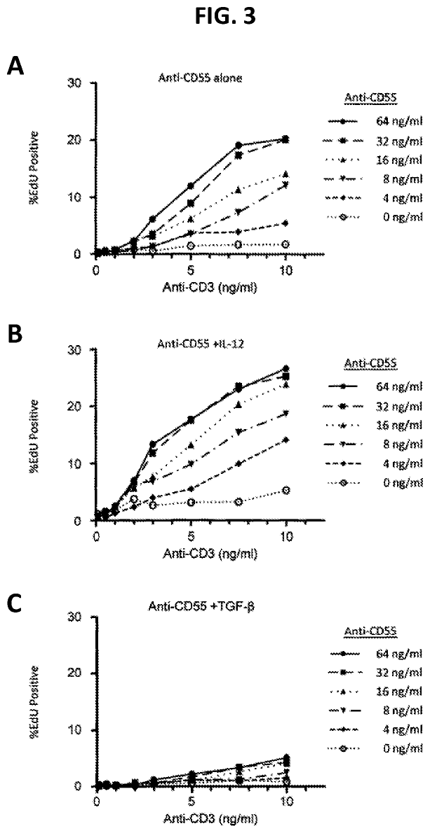

Human CD4+ T cells were enriched and cultured as described with regard to FIG. 2, panels A-C. Plates were treated with a matrix of serial dilutions comprising anti-CD3 between 10 ng/ml and 0.5 ng/ml, crossed with two-fold serial dilutions of anti-CD55 between 64 ng/ml and 4 ng/ml. Cells were then provided with no additional cytokines (FIG. 3, panel A), IL12 (FIG. 3, panel B), or TGF-β (FIG. 3, panel C). As shown in FIG. 3, panel B, the addition of IL-12 further increased T cell proliferation (compared to anti-CD55 alone, FIG. 3, panel A), while TGF-β repressed T cell proliferation. These results suggest that co-stimulation using a CD55-binding agent may be enhanced by IL-12, potentially by inducing proliferation of TH1 helper T cells. However, co-stimulation via a CD55-binding agent in and of itself does not overcome the effects of TGF-β, suggesting that CD55 co-stimulation is insufficient to drive Tconv proliferation within a tumor micro-environment high in TGF-β. Thus, administration of a CD55 binding agent along with a TGF-β inhibitor could be beneficial.

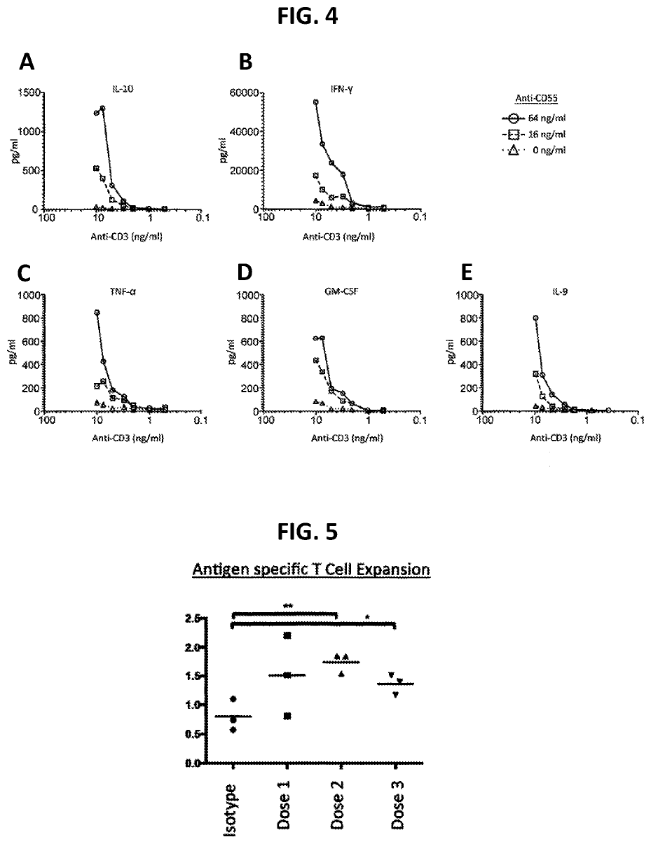

To further discern the phenotype of T cells co-stimulated by CD55, additional experiments were performed to evaluate cytokine production by proliferating CD4+ T cells. Human CD4+ T cells were enriched and cultured as described, and plates were treated with a matrix of serial dilutions comprising anti-CD3 between 10 ng/ml and 0.5 ng/ml, crossed with anti-CD55 at 64 ng/ml, 16 ng/ml, or 0 ng/ml. Following the proliferation period, supernatant was retrieved from each well and was assayed for the presence of five cytokines using the ProcartaPlex multiplex immunoassay (Affymetrix). Cytokines analyzed included IL-10 (FIG. 4, panel A), IFN-γ (FIG. 4, panel B), TNF-α (FIG. 4, panel C), GM-CSF (FIG. 4, panel D), and IL-9 (FIG. 4, panel E). All five cytokines showed dose-dependent production in response to anti-CD55, suggesting that a CD55-binding agent may induce cytokine production which may further amplify the proliferative effects of co-stimulation. For example, while the most highly produced cytokine was IFN-γ (FIG. 4, panel B), IL-10 secretion was also induced (FIG. 4, panel A). The production of TNF-α (FIG. 4, panel C), a strong pro-lysis cytokine, suggests that anti-CD55 co-stimulation could also promote tumor necrosis. The production of GM-CSF (FIG. 4, panel D) suggests that monocyte proliferation may be promoted by CD55 co-stimulation, thus potentially increasing trafficking of cells to the tumor which may then be activated by the presentation of tumor antigens. The production of IL-9, a T cell growth factor, suggests that CD55 co-stimulation may support T helper growth and survival even in the absence of antigen and/or antigen presenting cells. Together, these results suggest that CD55 co-stimulation may induce proliferation of multiple T cell types, and thus that a CD55 binding agent may be capable of producing a broad-spectrum immune response.

### Example 4—Effect of CD55-Binding Agents on Antigen-Specific T Cell Proliferation

To determine whether CD55-binding agents can stimulate proliferation of antigen-specific T cells, in vitro T cell proliferation assays were conducted using ovalbumin (OVA)-specific T cells. Murine DCs were loaded with purified ovalbumin (OVA). The loaded DCs were then presented to CFSE-labeled OT-1 transgenic T cells. Purified anti-CD3 and anti-CD55 or purified hamster anti-mouse IgG were added to the culture medium on day 3 (dose 1, isotype control) following the initiation of co-culture. A subset of T cell co-cultures were given a second dose of anti-CD3 and anti-CD55 on day 5, and a subset of those T cell co-cultures were given a third dose on day 7. Proliferation of T cells was assessed by dilution of CFSE on 10 days after T cell activation.

FIG. 5 is a graph indicating relative levels of OT-1 transgenic T cell proliferation induced by anti-CD55 and anti-CD3. T cells provided with a single dose of anti-CD3 and anti-CD55 showed a slight, though not statistically significant increase in proliferation. T cells provided with two or three doses did show significant increase in proliferation, though the third dose did not further increase proliferation over the second. These results suggest that a CD55-binding agent may augment an immune response to a specific tumor antigen by increasing the expansion of antigen-specific T cells.

As such, CD55-binding agents may be utilized as an immunotherapeutic agent either in vivo or in vitro. For example, naïve T cells may be isolated from a patient's peripheral blood and primed via cross-presentation of a tumor antigen by an antigen presenting cell in vitro. In some implementations, the cross-presentation may be performed in the presence of a CD55-binding agent in vitro in order to increase a population of activated T cells that may subsequently be transferred back to the patient intravenously. Additionally or alternatively, a therapeutically effective amount of a CD55-binding agent may be administered to a patient along with activated T cells that have been primed against a tumor-specific antigen.

### Example 5—Effect of CD55-Binding Agents on Tumor Growth

Historically, cancer vaccine-induced T cells have rarely been effective at eradicating tumors. The induced T cells, if they can recognize tumors and represent tumor-rejection antigens, often become sequestered, dissipate, undergo apoptosis, or merely fail to expand to levels capable of eradicating a tumor. Thus, while in vitro data may suggest that a targeted T cell marker may be effectively exploited to induce activation and/or proliferation, any induced T cells would still need to traffic to the site of the tumor, overcome any suppressive regulation, recognize tumor antigens, and effectively induce tumor cell death. As such, in vivo validation in a pre-clinical model is essential for establishing whether a theoretical potential therapy has practical value. For example, a target may be validated in model mammalian organism such as a mouse, rat, dog, primate, etc. and/or may be validated in human organisms. Assays for in vivo validation may include, but are not limited to, CD4+ and/or CD8+ T cell proliferation and/or cytokine production in TCR transgenic adoptive transfer and immunization systems, antigen feeding to promote in vivo induction of Treg cells, and/or tumor regression models, such as subcutaneous tumor transfer followed by vaccination.

As an example, murine in vivo tumor regression models may indicate whether a therapeutic agent effectively primes the mouse immune system against an implanted tumor. In a tumor cell line that does not express CD55, one can test the ability of a CD55-binding agent to enhance an immune response directed against the tumor in vivo. One such murine tumor cell line is MCA 205, a 3-methylcholanthrene-induced weakly immunogenic fibrosarcoma that is syngeneic to B6 mice.

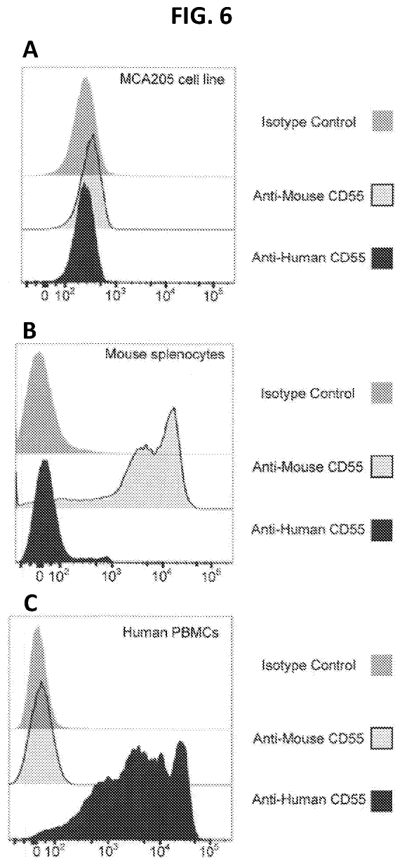

To demonstrate whether MCA 205 tumor cells express CD55 they were incubated with either labeled anti-mouse CD55 (Ultra-LEAF purified anti-mouse CD55; RIKO-3 clone; Biolegend; San Diego, Calif.); anti-human CD55, or an isotype control (hamster IgG). As shown in FIG. 6, panel A, neither anti-mouse CD55 nor anti-human CD55 was able to bind to MCA 205 cells significantly higher than the isotype control. In contrast, mouse splenocytes expressed CD55 as demonstrated by anti-mouse CD55 staining, as shown in FIG. 6, panel B. Human PBMCs were recognized by anti-human CD55, but not anti-mouse CD55, as shown in FIG. 6, panel C. These results indicate that the RIKO-3 anti-mouse CD55 antibody specifically binds mouse splenocytes, but does not bind to MCA 205 cells. As such, tumor regression resulting from CD55 binding agent therapy may be attributed to effects of the antibody acting on the immune system, and not to the antibody directly binding to tumor cells.

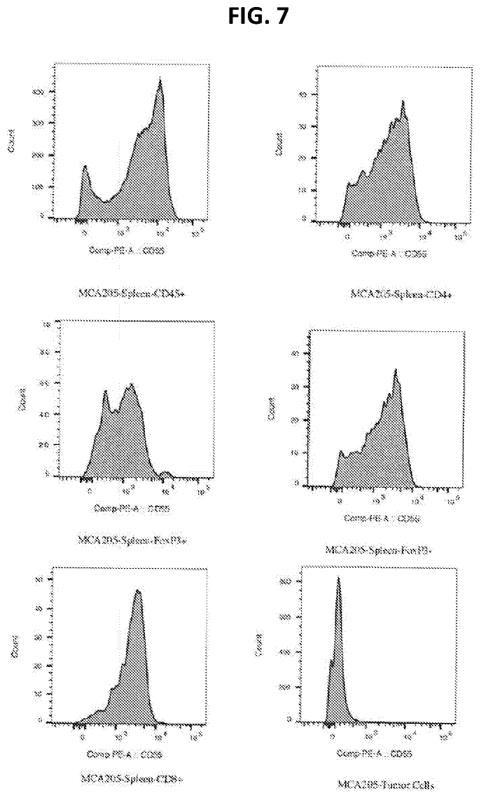

FIG. 7 shows CD55 protein expression on mouse splenocytes and MCA205 tumor cells from tumor-bearing mice. C57BL/6 mice were inoculated with MCA205 cells s.c.; 18 days later spleens and tumors were harvested and processed into single cell suspensions and stained for flow cytometry analysis directly ex vivo. The gating strategy included, lymphocyte gate, singlets and live cells. Cell surface CD55 protein expression was assessed on splenocytes, total CD45+(for hematopoietic cells), TCR-beta+(alpha/beta T cells), total CD4+ and CD8+(T cell subsets), CD4+CD25+Foxp3+(Tregs) and CD4+Foxp3− T conventionals. CD55 cell surface expression on tumor cells was assessed by gating on CD45− events in a tumor sample.

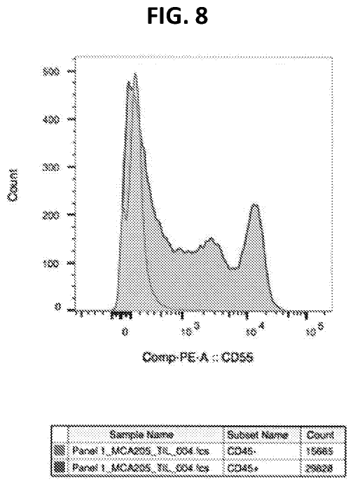

FIG. 8 shows CD55 protein expression on mouse hematopoietic cells and tumor cells from MCA205 tumors. C57BL/6 mice were inoculated with MCA205 cells s.c., 18 days tumors were harvested, enzyme digested, processed into single cell suspensions and stained for flow cytometry analysis directly ex vivo. The gating strategy included large light gate, singlets and live cells. Cell surface CD55 protein expression was assessed on total CD45+(for hematopoietic cells) and CD45− (for MCA205 tumor cells).

The ability of CD55-binding agent therapy in combination with anti-PD-1 to boost tumor immunotherapy and affect tumor growth was evaluated. In a first experiment, wild type (wt) C57BL/6 mice were subcutaneously implanted with 5×105 MCA-205 sarcoma tumor cells. Seven days later, and again after 9 days and 11 days, the mice were treated with 400 μg of Hamster IgG (Polyclonal)(Bio X Cell; West Lebanon, N.H.); 200 μg of Hamster IgG and 200 μg of anti-CD55 (Ultra-LEAF purified anti-mouse CD55; RIKO-3 clone) (Biolegend; San Diego, Calif.); 200 μg of Hamster IgG and 200 μg of anti-PD-1; or 200 μg of anti-CD55 and 200 μg of anti-PD-1. PD-1 (programmed cell death protein 1) is a T cell checkpoint protein that, when engaged by a ligand, inhibits signaling downstream of TCR. Further, PD-1 activity enhances both proliferation and suppressive activity of Treg cells. Therapeutic blockade of PD-1 with anti-PD-1 has been shown to stimulate tumor regression in both pre-clinical models and in clinical trials.

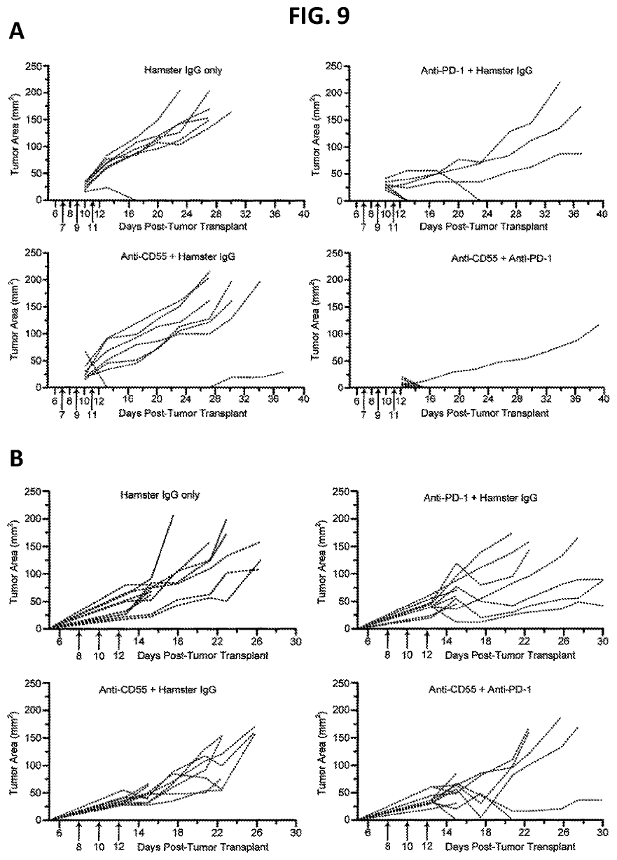

FIG. 9, panel A, shows graphs of tumor growth over time for each treatment group. Mice treated with either anti-CD55 or anti-PD-1 alone showed reduced tumor growth compared to control mice. When treated with both antibodies in tandem, a synergistic effect was seen, as combined treatment was more effective than treatment with either anti-CD55 or anti-PD-1 alone.

In a second experiment, tumors were transplanted into mice as described above. Mice were treated based on tumor size (30-60 mm2) on days 8, 10, and 12 post-transplant using the same clones and doses described above. Again, combined therapy with anti-CD55 and anti-PD-1 had greater efficacy than treatment with either antibody alone (FIG. 9, panel B). These results suggest that CD55-binding agents may provide a synergistic effect to anti-PD-1 therapy. Two anti-PD-1 variants have been FDA approved in the treatment of metastatic melanoma and lung cancer: pembrolizumab (Keytruda®) and nivolumab (Opdivo®). CD55-binding agents may enhance and/or boost anti-PD-1 therapy, thus allowing for therapy that results in more robust tumor regression, prolonged patient survival, and/or a broader range of applicable cancers and may work in combination with other forms of immunotherapy. As described with regard to in vitro experiments, patient T cells may be isolated and treated with CD55 binding agents in vitro prior to being transferred back to the patient. Such forms of T cell transfer may be accompanied by anti-PD-1 therapy and/or CD55-binding agent therapy.

### Example 6—Effects of CD55-Binding Agents on Human T Cell Proliferation

CD4 T cells were enriched from frozen PBMC with a CD4 T cell enrichment kit (StemCell) and CXCR5+ memory CD4 T cells were then sorted using the FACS Aria II. Cells were sorted using the following gating strategy: CD4+CD8-CD25-CD127+CD45RA- and CXCR5+. Sorted CXCR5+CD4 T cells were labeled with Cell Proliferation Dye eFluor 450 (eBioscience) and cultured together with sorted autologous naïve B cells labeled with CFSE (eBioscience) in the presence or absence of SEB at 0.1 μg/ml in a 96-well plate U-bottomed coated with increasing concentrations of isotype control or CD55 antibody (clone JS-11—BioLegend®). In some conditions, Treg cells labeled with Cell Proliferation Dye eFluor 670 (eBioscience) were added to the CD4 T cell/B cell coculture. After 4 days of culture, cells were harvested and proliferation was assessed for each cell population by flow cytometry. Cell proliferation was analyzed by measuring the frequency of cells that diluted the proliferation dye but also by determining absolute cell counts for each cell type.

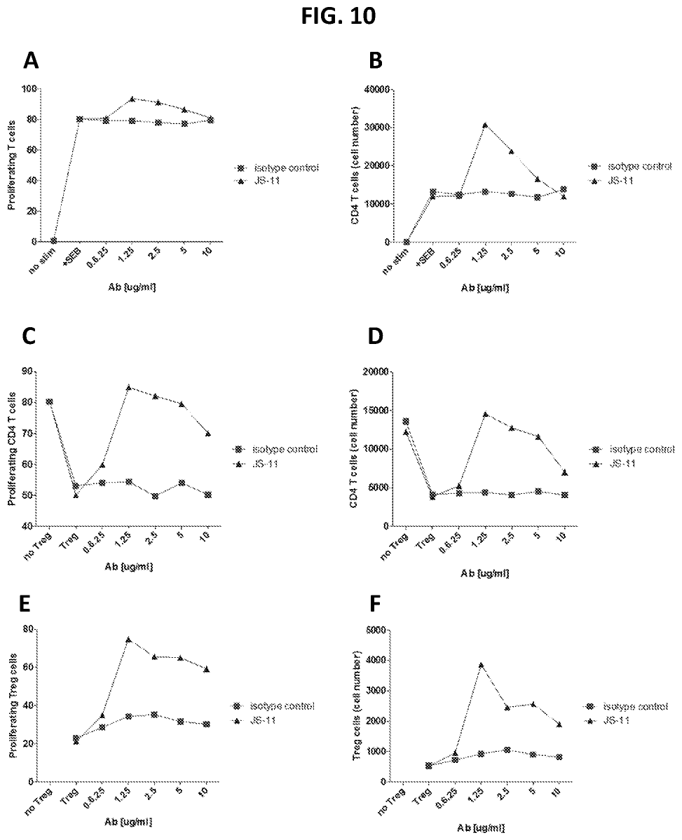

As shown in FIG. 10, CD55 costimulation on T cells had only a marginal effect in this assay, as the level of proliferation in the absence of CD55 was already very high. Interestingly, CD55 costimulation was capable of releasing the Treg-mediated inhibition on Tconv proliferation whereas the isotype control had no impact. This effect was observed albeit CD55 induced Treg cell proliferation in vitro. These results show that an antibody to CD55 can promote T cell proliferation.

### Example 7—Costimulation of Human CD4 T-Cell Proliferation

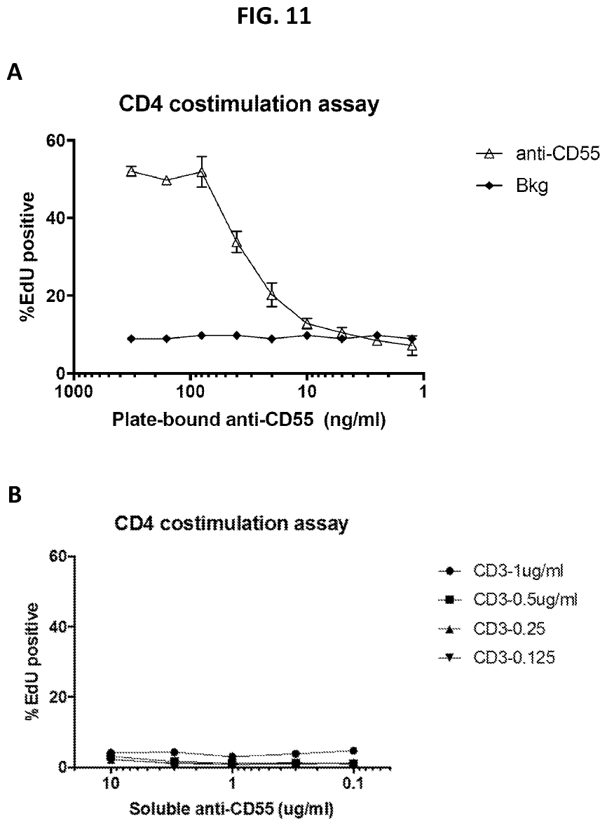

In this example, the effects of plate-bound and soluble anti-CD55 antibody on human CD4 T-cell proliferation was assessed. For the plate-bound condition, using goat anti-mouse IgG capture antibodies, mouse anti-human CD3 antibody was added to the plate at 2 ng/ml, a sub-threshold concentration for stimulation of CD4 T cell proliferation (background). Results are shown in FIG. 11 (panel A). Binding of mouse anti-human CD55 (in this example, the JS11 mouse anti-human CD55) to the plate stimulated proliferation of added human CD4 T cells in a dose-dependent manner. Both antibodies were plate-bound in this assay.

For the soluble anti-CD55 antibody condition, anti-CD3 antibody was bound directly to the plate at the concentrations indicated in FIG. 11, panel B. The plate was blocked with 1% BSA to prevent further attachment of antibodies to the plate. CD4 T cells were added in the presence of the indicated concentrations of mouse anti-human CD55 antibody (in this example, the JS11 mouse anti-human CD55 antibody). In this assay, only the anti-CD3 antibody was plate-bound. Results are shown in FIG. 11, panel B. The presence of soluble CD55 antibody did not enhance the proliferation of CD4 T cells exposed to anti-CD3 at the concentrations indicated.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of treating a cell proliferative disorder, comprising:
administering to a subject having a cell proliferative disorder a therapeutically effective amount of a CD55-binding agent,
wherein at the time of the administering, abnormally proliferating cells of the cell proliferative disorder are not suspected of exhibiting overexpression of CD55.

2. The method according to claim 1, wherein at the time of the administering, it has been determined that abnormally proliferating cells of the cell proliferative disorder do not overexpress CD55.

3. The method according to claim 2, comprising determining that abnormally proliferating cells of the cell proliferative disorder do not overexpress CD55.

4. The method according to any one of claims 1 to 3, wherein the subject to whom the CD55-binding agent is administered is receiving an antibody therapy.

5. The method according to claim 4, wherein the antibody therapy is being administered to the subject to treat the cell proliferative disorder by inducing antibody-dependent cellular cytotoxicity (ADCC), and wherein the CD55-binding agent is administered to the subject to potentiate ADCC of the antibody therapy.

6. A method of treating a cell proliferative disorder, comprising:
administering to a subject having a cell proliferative disorder a therapeutically effective amount of a CD55-binding agent,
wherein the CD55-binding agent is administered to the subject to enhance a T cell response to abnormally proliferating cells of the cell proliferative disorder.

7. The method according to claim 6, wherein the subject to whom the CD55-binding agent is administered is receiving an antibody therapy.

8. The method according to claim 7, wherein the antibody therapy is being administered to the subject to treat the cell proliferative disorder by inducing antibody-dependent cellular cytotoxicity (ADCC), and wherein the CD55-binding agent is administered to the subject to potentiate the ADCC of the antibody therapy.

9. The method according to any one of claims 1 to 8, wherein the CD55-binding agent is a small molecule.

10. The method according to any one of claims 1 to 8, wherein the CD55-binding agent is a peptide or polypeptide.

11. The method according to claim 10, wherein the CD55-binding agent is a CD55 ligand.

12. The method according to claim 10, wherein the CD55-binding agent is an antibody that specifically binds CD55.

13. The method according to claim 12, wherein the antibody that specifically binds CD55 is selected from the group consisting of: an IgG, Fv, scFv, Fab, F(ab′)₂, and Fab′.

14. The method according to any one of claims 1 to 13, further comprising administering to the subject a T cell activator.

15. The method according to claim 14, wherein the T cell activator is an immune checkpoint inhibitor.

16. The method according to claim 15, wherein the immune checkpoint inhibitor is an agonist of a T cell co-stimulatory receptor.

17. The method according to claim 15, wherein the immune checkpoint inhibitor is an antagonist of a T cell inhibitory signal.

18. The method according to claim 15, wherein the immune checkpoint inhibitor is selected from the group consisting of: a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, an OX40 agonist, a glucocorticoid-induced TNFR-related protein (GITR) agonist, a CD137 agonist, and a CD40 agonist.

19. The method according to claim 14, wherein the T cell activator is a cytokine.

20. The method according to claim 14, wherein the T cell activator is an antagonist of an inhibitory immune receptor.

21. The method according to any one of claims 14 to 20, wherein the CD55-binding agent and the T cell activator are administered concurrently.

22. The method according to any one of claims 14 to 20, wherein the CD55-binding agent and the T cell activator are administered sequentially.

23. The method according to any one of claims 1 to 22, wherein the cell proliferative disorder is cancer.

24. A method of treating a cell proliferative disorder, comprising:
administering to a subject having a cell proliferative disorder:
a therapeutically effective amount of a CD55-binding agent; and
a therapeutically effective amount of a T cell activator.

25. The method according to claim 24, wherein at the time of the administering, abnormally proliferating cells of the cell proliferative disorder are not suspected of exhibiting overexpression of CD55.

26. The method according to claim 25, wherein at the time of the administering, it has been determined that abnormally proliferating cells of the cell proliferative disorder do not overexpress CD55.

27. The method according to claim 26, comprising determining that abnormally proliferating cells of the cell proliferative disorder do not overexpress CD55.

28. The method according to any one of claims 24 to 27, wherein the subject to whom the CD55-binding agent is administered is receiving an antibody therapy.

29. The method according to claim 28, wherein the antibody therapy is being administered to the subject to treat the cell proliferative disorder by inducing antibody-dependent cellular cytotoxicity (ADCC), and wherein the CD55-binding agent is administered to the subject to potentiate ADCC of the antibody therapy.

30. The method according to any one of claims 24 to 29, wherein the CD55-binding agent is administered to the subject to enhance a T cell response to abnormally proliferating cells of the cell proliferative disorder.

31. The method according to any one of claims 24 to 30, wherein the CD55-binding agent is a small molecule.

32. The method according to any one of claims 24 to 30, wherein the CD55-binding agent is a peptide or polypeptide.

33. The method according to claim 32, wherein the CD55-binding agent is a CD55 ligand.

34. The method according to claim 32, wherein the CD55-binding agent is an antibody that specifically binds CD55.

35. The method according to claim 34, wherein the antibody that specifically binds CD55 is selected from the group consisting of: an IgG, Fv, scFv, Fab, F(ab′)₂, and Fab′.

36. The method according to any one of claims 24 to 35, wherein the T cell activator is an immune checkpoint inhibitor.

37. The method according to claim 36, wherein the immune checkpoint inhibitor is an agonist of a T cell co-stimulatory receptor.

38. The method according to claim 36, wherein the immune checkpoint inhibitor is an antagonist of a T cell inhibitory signal.

39. The method according to claim 36, wherein the immune checkpoint inhibitor is selected from the group consisting of: a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, an OX40 agonist, a glucocorticoid-induced TNFR-related protein (GITR) agonist, a CD137 agonist, and a CD40 agonist.

40. The method according to any one of claims 24 to 35, wherein the T cell activator is a cytokine.

41. The method according to any one of claims 24 to 35, wherein the T cell activator is an antagonist of an inhibitory immune receptor.

42. The method according to any one of claims 24 to 41, wherein the CD55-binding agent and the T cell activator are administered concurrently.

43. The method according to any one of claims 24 to 41, wherein the CD55-binding agent and the T cell activator are administered sequentially.

44. The method according to any one of claims 24 to 43, wherein the cell proliferative disorder is cancer.

45. A pharmaceutical composition, comprising:
a CD55-binding agent;
a T cell activator; and
a pharmaceutically acceptable excipient.

46. The pharmaceutical composition of claim 45, wherein the CD55-binding agent is a small molecule.

47. The pharmaceutical composition of claim 45, wherein the CD55-binding agent is a peptide or polypeptide.

48. The pharmaceutical composition of claim 47, wherein the CD55-binding agent is a CD55 ligand.

49. The pharmaceutical composition of claim 47, wherein the CD55-binding agent is an antibody that specifically binds CD55.

50. The pharmaceutical composition of 49, wherein the antibody that specifically binds CD55 is selected from the group consisting of: an IgG, Fv, scFv, Fab, F(ab′)2, and Fab′.

51. The pharmaceutical composition of any one of claims 45 to 50, wherein the T cell activator is an immune checkpoint inhibitor.

52. The pharmaceutical composition of any one of claim 51, wherein the immune checkpoint inhibitor is an agonist of a T cell co-stimulatory receptor.

53. The pharmaceutical composition of any one of claim 51, wherein the immune checkpoint inhibitor is an antagonist of a T cell inhibitory signal.

54. The pharmaceutical composition of claim 53, wherein the immune checkpoint inhibitor is selected from the group consisting of: a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, an OX40 agonist, a glucocorticoid-induced TNFR-related protein (GITR) agonist, a CD137 agonist, and a CD40 agonist.

55. The pharmaceutical composition of any one of claims 45 to 50, wherein the T cell activator is a cytokine.

56. The pharmaceutical composition of any one of claims 45 to 50, wherein the T cell activator is an antagonist of an inhibitory immune receptor.

57. A kit, comprising:
a pharmaceutical composition comprising a CD55-binding agent; and
instructions for administering the pharmaceutical composition to a subject having a cell proliferative disorder.

58. The kit of claim 57, wherein the pharmaceutical composition further comprises a T cell activator.

59. A kit, comprising:
the pharmaceutical composition of any one of claims 45 to 56; and
instructions for administering the pharmaceutical composition to a subject having a cell proliferative disorder.

60. The kit of any one of claims 57 to 59, wherein the kit comprises the pharmaceutical composition in one or more unit dosages.

61. The kit of any one of claims 57 to 59, wherein the kit comprises the pharmaceutical composition in two or more unit dosages.

62. The kit of any one of claims 57 to 61, comprising instructions for administering the pharmaceutical composition to a subject having a cell proliferative disorder in which abnormally proliferating cells of the cell proliferative disorder are not suspected of exhibiting overexpression of CD55.

63. The kit of any one of claims 57 to 62, comprising instructions for administering the pharmaceutical composition to a subject receiving an antibody therapy.

64. The kit of claim 63, wherein the antibody therapy is being administered to the subject to treat the cell proliferative disorder by inducing antibody-dependent cellular cytotoxicity (ADCC), and wherein the instructions are for administering the CD55-binding to the subject to potentiate ADCC of the antibody therapy.

65. A kit, comprising:
a CD55-binding agent;
a T cell activator; and
instructions for administering the CD55 binding agent and T cell activator to a subject having a cell proliferative disorder.

66. The kit of claim 65, wherein the kit comprises the CD55-binding agent and the T cell activator in one or more unit dosages.

67. The kit of claim 65, wherein the kit comprises the CD55-binding agent and the T cell activator in two or more unit dosages.

68. The kit of any one of claims 65 to 67, wherein the CD55-binding agent and T cell activator are present in separate containers.

69. The kit of any one of claims 65 to 68, wherein the instructions are for administering the CD55-binding agent and the T cell activator to a subject having a cell proliferative disorder in which abnormally proliferating cells of the cell proliferative disorder are not suspected of exhibiting overexpression of CD55.

70. The kit of any one of claims 65 to 69, comprising instructions for administering the CD55-binding agent and the T cell activator to a subject receiving an antibody therapy.

71. The kit of claim 70, wherein the antibody therapy is being administered to the subject to treat the cell proliferative disorder by inducing antibody-dependent cellular cytotoxicity (ADCC), and wherein the instructions are for administering the CD55-binding agent and the T cell activator to the subject to potentiate ADCC of the antibody therapy.

72. The kit of any one of claims 57 to 71, wherein the CD55-binding agent is a small molecule.

73. The kit of any one of claims 57 to 71, wherein the CD55-binding agent is a peptide or polypeptide.

74. The kit of claim 73, wherein the CD55-binding agent is a CD55 ligand.

75. The kit of claim 73, wherein the CD55-binding agent is an antibody that specifically binds CD55.

76. The kit of claim 75, wherein the antibody that specifically binds CD55 is selected from the group consisting of: an IgG, Fv, scFv, Fab, F(ab′)₂, and Fab′.

77. The kit of any one of claims 58 to 76, wherein the T cell activator is an immune checkpoint inhibitor.

78. The kit of claim 77, wherein the immune checkpoint inhibitor is an agonist of a T cell co-stimulatory receptor.

79. The kit of claim 77, wherein the immune checkpoint inhibitor is an antagonist of a T cell inhibitory signal.

80. The kit according to claim 77, wherein the immune checkpoint inhibitor is selected from the group consisting of: a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, an OX40 agonist, a glucocorticoid-induced TNFR-related protein (GITR) agonist, a CD137 agonist, and a CD40 agonist.

81. The kit of any one of claims 58 to 76, wherein the T cell activator is a cytokine.

82. The kit of any one of claims 58 to 76, wherein the T cell activator is an antagonist of an inhibitory immune receptor.

83. The kit of any one of claims 57 to 80, wherein the cell proliferative disorder is cancer.
